(19) United States
(12) Patent Application Publication
(10) Pub. No.: US 2011/0045558 A1
(43) Pub. Date: Feb. 24, 2011

(54) PROCESS FOR THE PREPARATION OF CITRIC ACID EMPLOYING FILAMENTOUS FUNGI IN A CULTURE MEDIUM COMPRISING GLYCEROL

(76) Inventors: Hugo Marc Karel Bauweleers, Boutersem (BE); Dominique Robert Groeseneken, Hoegaaden (BE)

Correspondence Address: NIXON & VANDERHYE, PC, 901 NORTH GLEBE ROAD, 11TH FLOOR, ARLINGTON, VA, 22203, US

(21) Appl. No.: 12/528,546

(22) PCT Filed: Mar. 6, 2008
(86) PCT No.: PCT/EP08/52717

§ 371 Date: Nov. 10, 2010

(30) Foreign Application Priority Data
Mar. 8, 2007 (EP) 07103730.3

(51) Int. Cl. C12P 7/48; C07C 29/94

(52) U.S. Cl. 435/144; 568/869

(57) ABSTRACT

The present invention relates to a method for producing citric acid from glycerol with a yield of more than 70%. The method comprises fermenting a filamentous fungus on a substrate which comprises glycerol. The advantage of the method according to the invention is that a widely available substrate, i.e. glycerol, can be used and that yields may be obtained which are much higher than the yields obtained using state of the art methods for producing citric acid from glycerol. The glycerol is used in combination with one or more other carbon sources.

### FIELD OF THE INVENTION

The present invention relates to a method for producing citric acid. In particular, it relates to the production of citric acid by fermentation.

### BACKGROUND OF THE INVENTION

Citric acid (2-hydroxy-propane-1,2,3-tricarboxylic acid) is known as an industrially important organic acid which is used e.g. as food additive, preservative or as stabilizator of oils and fats due to its ability to complex heavy metal ions like copper and iron. Originally, it has been isolated from citrus plants. Chemical synthesis of citric acid is also possible, however, not at all suitable for industrial production due to the expensive raw materials and a complicated process with low yield.

Therefore, over the past decades, other approaches to manufacture citric acid using microbial conversions, which would be more economical as well as ecological, have been investigated.

Citric acid production from a number of substrates including glucose or sucrose has been reported in several microorganisms, such as fungi including yeasts, using different cultivation methods. Examples of known fungi able to directly produce citric acid include, for instance, strains from the genera of Aspergillus, in particular A. niger, or yeasts such as Yarrowia, in particular Yarrowia lipolytica.

U.S. Pat. No. 3,773,620 describes the use of n-parafins as raw material for citric acid production in combination with saturated alcohols. In this process also isocitric acid is formed. Certain compounds (like monofluoroacetic acid) are necessary to obtain an acceptable yield of citric acid in the process.

U.S. Pat. No. 3,801,455 describes the production of citric acid from n-parafins but also from other carbon sources, such as glycerol, to produce citric acid with a yield of about 50-55% of the theoretical maximum on n-parafin. With glucose as raw material, a yield of about 63% of the theoretical maximum is reached. Additional compounds, for example monofluoroacetic acid, are necessary to obtain an acceptable yield of citric acid in the process.

Rymowicz (2006) describes citric acid production on raw glycerol by Yarrowia yeasts, with a yield of around 62%.

### DETAILED DESCRIPTION OF THE INVENTION

The present invention relates to a method for producing citric acid from glycerol with a yield of more than 70% w/w, which method comprises fermenting a filamentous fungus on a substrate which comprises 10-90% w/w glycerol and 90-10% of another carbon source, both expressed as glucose equivalents on the total carbohydrate content of the substrate.

One advantage of the method according to the invention is that a widely available substrate, i.e. glycerol, can be used. A main source of glycerol is the production of biodiesel. Another advantage is that yields may be obtained which are much higher than obtained using state of the art methods for producing citric acid from glycerol.

The glycerol is combined with another carbon source, such as with the standard fermentation substrates for citric acid production, like molasses, glucose, fructose, sucrose, polyalcohols, starch hydrolysate and starch containing substrates, such as corn and cassaya. In case of molasses, beet or cane molasses may be used. The carbohydrates may be in solid form, in liquid form, liquefied form or as a syrup, such as for instance liquefied corn, starch or glucose, fructose, sucrose or molasses syrup. The carbohydrates may also be used in combination. The glycerol mixed with one or more other carbon sources makes up 100% of the carbohydrate content of the substrate. Depending on the fermentation conditions and the strains used, the carbohydrates to be mixed with glycerol may vary.

In the context of the present invention, the phrase ‘production by fermentation’ refers to the production of citric acid by a microorganism, in particular a filamentous fungus, from a carbohydrate source by means of one or more biological conversion steps, without the need of any additional chemical conversion steps. The fermentation medium may be any suitable medium for the production of citric acid. Typically, the medium is an aqueous medium comprising for instance salts, carbohydrates, nutrients and a certain pH. Fermentative production of citric acid on industrial scale is well-known in the art, see for example U.S. Pat. No. 5,081,025.

The fermentations are typically performed in batch, fed-batch or continuous mode. Both submerged and surface fermentation are encompassed by the invention.

In case of submerged fermentation, the preferred carbohydrate to be mixed with glycerol is selected from glucose or sucrose syrups or liquefied starches. In one embodiment, citric acid is produced via submerged fermentation starting from a carbohydrate raw material such as for instance cassaya or corn, which may be milled and mixed with water. A seed fermentation may be prepared in a separate fermentor. The liquefaction of the starch may be performed in the presence of an amylolytic enzyme such as for instance amylases, cellulases, lactases or maltases. For the main fermentation, the concentration of carbohydrates in the mix may be in the range of about 150 to 250 g/l, preferably in the range of about 150-2000, more preferably in the range of about 150-180 g/l.

In case of surface fermentation, the preferred carbohydrate to be mixed with glycerol is selected from molasses, sucrose syrup or a solid substrate containing carbohydrates (the so called solid state fermentation). In one embodiment, citric acid is produced via surface fermentation starting from a carbohydrate raw material such as for instance a mix of beet and cane molasses or sucrose. For both submerged and surface fermentation, additives, such as salts (e.g. hexacyanoferrate), nutrients and antifoam may be added before or during fermentation.

The glycerol may be mixed with another carbon source to obtain an end concentration of glycerol in the range of 10 to 90% w/w, preferably in the range of 10 to 85% w/w, 10 to 80% w/w, 10 to 75% w/w, 10 to 70% w/w, 10 to 65% or 10 to 60%, more preferably in the range of 10 to 55% w/w, 10 to 50% w/w, 10 to 45% w/w or 10 to 40% w/w, most preferably, in the range of 10 to 35% w/w, 10 to 30% w/w, 10 to 25% w/w or 10-30% w/w. In one embodiment of the invention, glycerol is mixed with another carbon source in an end concentration of 10-30% w/w for surface fermentation. In another embodiment, glycerol is mixed with another carbon source in an end concentration of 10 to 50% w/w for submerged fermentation.

All these percentages are expressed as glucose-equivalents, whereby 100 gram glycerol equals 97.82 gram glucose-equivalents. Therefore, the phrase ‘10% w/w glycerol expressed as glucose equivalents’ refers to 10 gram glucose-equivalents glycerol per 100 g glucose equivalents expressed as percentage of the total carbohydrate content of the substrate.

Any filamentous fungus may be used in the method according to the invention. Preferably a filamentous fungus with GRAS status (generally recognized as safe) is used, more preferably an Aspergillus selected from A. niger, A. awamori, A. aculeatus, A. japonicus, A. oryzae, A. vadensis, A. carbonarius, A. tubingensis, A. lacticoffeatus, A. brasiliensis, A. piperis A. costaricaensis or A. foetidus is used. Even more preferably, an A. foetidus var acidus, A. foetidus var pallidus, or an A. niger var awamori is used. Yet even more preferably an A. niger ATCC1015 is used and most preferably A. niger CBS 513.88.

In the context of the present invention, the term ‘glycerol’ refers to 1,2,3-propanetriol and the term is interchangeably used with the term ‘glycerin’.

In the context of the present invention, the term ‘citric acid’ refers to any chemical form of citric acid found in aqueous solutions, such as for instance un-dissociated, in its free acid form or dissociated as an anion. The solubilized salt form of citric acid may be characterized as the anion in the presence of any kind of cations usually found in fermentation supernatants, such as for instance potassium, sodium, calcium or ammonium. Also included are isolated crystals of the free acid form of citric acid. On the other hand, isolated crystals of a salt form of citric acid are called by their corresponding salt name, i.e. sodium citrate, potassium citrate, calcium citrate and the like.

In a further aspect, the process for the production of citric acid as described above may be combined with further steps of separation or purification of the produced citric acid from other components in the fermentation broth, i.e. so-called downstream processing steps. These steps may include any means known to a skilled person, such as, for instance, concentration, crystallization, precipitation, adsorption, ion exchange, electrodialysis, bipolar membrane electrodialysis and/or reverse osmosis.

The citric acid may be converted for instance into monosodium citrate, trisodium citrate, tricalcium citrate, trisodium citrate dihydrate, tripotassium citrate, monosodium citrate anhydrous, or crystallized as citric acid anhydrous or citric acid monohydrate.

Citric acid and its salts as produced by a method described herein may be further used as ingredient or additive for e.g. food (such as e.g. bakery products, baby food, fats and oils, sweets, cheese products, dairy products), beverages such as e.g. carbonated soft drinks, syrups, fruit juices and drinks, wines, ready-to-drink teas), pharmaceuticals (such as e.g. tablets, syrups, suspensions/solutions), cleaners and detergents (such as e.g. deodorant soap, dish washing liquids/powders), in personal care products (such as e.g. shampoos, creams and lotions, hygiene products, toothpastes) or in other industrial applications such as in adhesives, animal feed, photo chemicals and the like.

According to the method of the invention, citric acid is produced in a yield of at least 70% w/w, which means that at least 70 gram of citric acid monohydrate is formed per 100 gram of glucose equivalents in the substrate. More preferably, the citric acid yield is at least 75%, 80%, 85% or 90% w/w. Even more preferably, the citric acid yield is at least 92%, 94%, 95%, 96%, 97%, 98%, 99% w/w. Most preferably, the citric acid yield is at least 100%, 103%, 106%, 109% or 112% w/w.

The glycerol which is used in the method of the invention may be crude, i.e. raw and unpurified glycerol, or it may be pre-treated to remove contaminants. Methods for purifying crude glycerol are known in the art, for example from EP 0 358 255, which describes the purification of raw glycerol by microfiltration.

The use of purified glycerol is particularly advantageous in surface fermentation where it allows for the use of a higher concentration of glycerol compared to the situation when raw glycerol is used. In one embodiment, 80-90% purified glycerol (expressed as glucose equivalents) is used for citric acid production by surface fermentation.

In one embodiment of the present invention, a simple filtration technique is used to purify raw glycerol to a quality which is acceptable for surface fermentation use. This process comprises mixing crude glycerol with CaO in a final concentration of at least 2.0 g CaO per kg of the mixture and allowing it to react, followed by filtering the reaction mixture over a pressure filter to obtain purified glycerol.

To the applicant's knowledge, this simple and straightforward process for purifying crude glycerol is novel. Therefore, in another aspect of the invention, the invention relates to a process to purify crude glycerol by mixing the crude glycerol with CaO in a final concentration of at least 2.0 g CaO per kg of the mixture and allowing it to react, followed by filtering the reaction mixture over a pressure filter to obtain purified glycerol.

The CaO used in the present invention may be in any suitable for, be it powder or liquid. In one embodiment of the present invention, CaO in the form of lime milk is used. The CaO is mixed with the glycerol to obtain a final concentration of at least 2.0 CaO, preferably the final concentration is at least 2.5 gram CaO per kg of the mixture, more preferably it is at least 3.0 or 3.5 gram CaO per kg of the mixture. Typically, it will not be more than 10 gram CaO per kg of the mixture.

The purification method according to the invention allows for short reaction times. The reaction time may be as short as less than 120 minutes. Preferably, it is less than 90, 80, 70 or 60 minutes, more preferably, it is less than 50, 40, 30, 20 or 10 minutes. Most preferably, the reaction time is less than 8, 6, 5, 4, 3, 2 or 1 minute. Of course, longer reaction times may be applied, but are not necessary and will reduce the efficiency of the purification process.

In the method according to the invention, the reaction mixture of glycerol and CaO is purified to remove impurities. The filter used to filter the reaction mixture may be a very simple filter, such as a pressure filter or a vacuum filter. There is no need to use a microfiltration unit or a ceramic filter. The filter may have a pre-coat or body feed applied to it. Suitable filter aids to be used in body feed filtration include silicates, such as perlite, an amorphous mineral consisting of fused sodium potassium aluminium silicate, produced by e.g. Dicalite Europe NV, Belgium; gypsum, which is for example produced in the citric acid downstream processing; kieselguhr, also called diatomeous earth, produced by e.g. Eagle Picher Inc., USA, brand name Celatom®. In one embodiment, a Dicalite® filter aid is used.

### EXAMPLES

### Example 1

### Production of Citric Acid by Surface Fermentation Using a Mixture of Molasses and Crude (Unpurified) Glycerol by A. niger

A mixture of beet and cane molasses was diluted with demineralized water to obtain 240 g sucrose per liter. Raw glycerol was diluted to 260 g/L expressed as glycerol. The two solutions were mixed in a ratio of 70% w/w molasses and 30% w/w of glycerol, both expressed as glucose-equivalents on the total carbohydrate content. To this mixture 3 ml phosphoric acid 5%, 0.7 g Na₄Fe(CN)₆.10H₂O, 0.45 g powdered activated carbon and 1.0 mg Zn as zinc sulphate was added per litre of mixture. The pH was adjusted to 6.15 with sulphuric acid and the mixture was put in a tray with a depth of 10 cm. This tray was pasteurized at 70° C. and allowed to cool down to 40° C. Spores of A. niger were added to the media and the trays were incubated in a climate room at a temperature of 35° C. and a relative humidity of at least 70%. Under these conditions the fermentation was blocked due to an insufficient formation of fungal mycelium. Further experiments revealed that fermentation was not blocked if much lower amounts of glycerol were used. On the medium without glycerol (but containing the molasses as carbohydrate) mycelium grew well and citric acid yield was as expected.

This example shows that raw (unpurified) glycerol will only work for surface fermentation if very small amounts of glycerol are used.

### Example 2

Production of Citric Acid by Surface Fermentation Using a Mixture of Molasses and Pretreated Crude Glycerol by A. niger

A mixture of beet and cane molasses was diluted with demineralized water to obtain 240 g sucrose per liter. Raw glycerol was pretreated by mixing it with lime milk to obtain 3 g CaO/kg of solution and stirring it during 5 minutes. It was then diluted with water and filtered in a pressure filter at 2 bar with a Dicalite filter aid. The resulting purified glycerol was diluted to 260 g/L expressed as glycerol. The two solutions were mixed in a ratio of 70% w/w molasses and 30% w/w of glycerol, both expressed as glucose-equivalents on the total carbohydrate content. To this mixture 3 ml phosphoric acid 5%, 0.7 g Na₄Fe(CN)₆.10H₂O, 0.45 g powdered activated carbon and 1.0 mg Zn as zinc sulphate was added per litre of mixture. The pH was adjusted to 6.15 with sulphuric acid and the mixture was put in a tray with a depth of 10 cm. This tray was pasteurized at 70° C. and allowed to cool down to 40° C. Spores of A. niger were added to the media and the trays were incubated in a climate room at a temperature of 35° C. and a relative humidity of at least 70%. The mycelium was cultivated to form a layer on the surface of the liquid and sucrose from the liquid was converted to citric acid. Fermentation was stopped when the sucrose, glucose, fructose and glycerol concentration had dropped below 4 g/l as measured by HPLC. After pasteurization to stop enzymatic activity, the citric acid concentration in the liquid was measured by HPLC or titration. Production performance was calculated as the yield of citric acid expressed as grams of citric acid monohydrate per 100 grams of glucose equivalents in the substrate. The yield in the fermentation with a mixture of raw materials was 95%. For comparison, the yield in a fermentation based on molasses alone was 100%.

This example shows that with pre-treated glycerol as substrate yields can be obtained in surface fermentation which are comparable to the yields obtained with molasses as substrate.

### Example 3

### Production of Citric Acid by Submerged Fermentation Using a Mixture of Corn, Cassaya and Crude Glycerol by A. niger

The carbohydrate raw material, e.g. cassaya and corn, were milled and mixed with water. A seed fermentation was started in a separate fermentor, containing a slurry of corn flour as carbohydrate, liquefied with an amylolytic enzyme at a temperature of 90° C. After cooling to 37° C., spores of A. niger were brought in the fermentor and the seed fermentation was carried out with an air flow rate of 0.1 to 0.2 volume per volume per minute (vvm) and the temperature was controlled at 37° C. After approximately 20 hours, the seed fermentor content was transferred to the main fermentor. The main fermentor was prepared with a mix of 20% w/w corn flour, 70% w/w cassava flour and 10% w/w raw glycerol without pre-treatment (all expressed as glucose equivalents on the total carbohydrate content) by adding water, additives and nutrients. The total carbohydrate concentration in the mix was 150 g glucose-equivalents/l. An amylolytic enzyme and antifoam were added. After heating to 90° C. to liquefy the starch and subsequently cooling to 35° C., the content of a seed fermentor was transferred to the main fermentor. After the transfer, the fermentation was controlled by cooling at an air flow rate of 0.1 vvm and stopped when the carbohydrate was consumed, which took 140 hours. The citric acid concentration in the liquid was measured by HPLC or titration. The yield of citric acid (grams of citric acid monohydrate per 100 g of carbohydrate, expressed as glucose-equivalents) was 88% as compared to a yield of 85.5% which was obtained in a separate experiment using a medium without glycerol.

This example shows that the yields obtained with raw glycerol as substrate are similar to the yields obtained with corn and cassaya based media.

### Example 4

### Production of Citric Acid by Submerged Fermentation Using a Mixture of Corn, Cassava and Crude Glycerol by A. niger

This experiment was conducted as Example 3, only the carbohydrate content was made up of 20% w/w raw glycerol without pre-treatment, 60% w/w cassava flour and 20% w/w corn flour (all expressed as glucose equivalents on the total carbohydrate content). The yield of citric acid was 85% (grams of citric acid monohydrate per 100 g of carbohydrate, expressed as glucose-equivalents).

This example shows again that the yields obtained with raw glycerol as substrate are similar to the yields obtained with corn and cassaya based media.

### Example 5

### Production of Citric Acid by Submerged Fermentation Using a Mixture of Glucose Syrup and Crude Glycerol by A. niger

Glucose syrup is used as the carbohydrate raw material, mixed with water. A seed fermentation is started in a separate fermentor, containing a slurry of corn flour as carbohydrate, liquefied with an amylolytic enzyme at a temperature of 90° C. After cooling to 37° C., spores of A. niger are brought in the fermentor and the seed fermentation is carried out with an air flow rate of 0.1 to 0.2 volume per volume per minute (vvm) and the temperature is controlled at 37° C. After approximately 20 hours, the seed fermentor contents is transferred to the main fermentor. The main fermentor is prepared with a mix of 80% w/w glucose syrup, 20% raw glycerol without pre-treatment (both expressed as glucose equivalents on the total carbohydrate content), water, additives and nutrients. The concentration of carbohydrate in the mix is 160 g/l and antifoam is added. After heating to 120° C. to sterilise and subsequently cooling to 35° C., the content of the seed fermentor is transferred to the main fermentor. After the transfer, the fermentation is controlled by cooling at an air flow rate of 0.1 vvm and stopped when the carbohydrate is consumed, which typically takes about 80-100 hours. The citric acid concentration in the liquid is measured by HPLC or titration. The yield of citric acid will be at least 90% of the yield which is obtained with the medium without glycerol.

1. A method for producing citric acid from glycerol with a yield of more than 70% w/w, which method comprises fermenting a filamentous fungus on a substrate which substrate comprises 10-90% w/w glycerol and 90-10% w/w of another carbon source, both expressed as glucose-equivalents on the total carbohydrate content of the substrate.

2. A method according to claim 1 wherein the another carbon source is molasses, glucose, fructose, sucrose, a polyalcohol, starch, a starch hydrolysate and starch containing substrates, or a combination thereof, in solid form, in liquid form, in liquefied form or as a syrup.

3. A method according claim 2, wherein the filamentous fungus is an Aspergillus species.

4. A method according to claim 2, wherein the Aspergillus species is an A. niger, A. awamori, A. aculeatus, A. japonicus, A. oryzae, A. vadensis, A. carbonarius, A. tubingensis, A. lacticoffeatus, A. brasiliensis, A. piperis A. costaricaensis or A. foetidus.

5. A method according to claim 4 wherein the Aspergillus is an A. niger, preferably A. niger as deposited under accession no. ATCC 1015 or under CBS 513.88.

6. A method according to claim 1, wherein the citric acid is produced by submerged fermentation.

7. A method according to claim 1, wherein the citric acid is produced by surface fermentation.

8. A method according to claim 1, wherein the glycerol is pre-treated before it is used in the production of citric acid.

9. A method according to claim 8, wherein the pre-treatment comprises mixing the glycerol with CaO in a final concentration of at least 2.0 g CaO per kg of the mixture and allowing it to react, followed by filtering the reaction mixture over a pressure filter to obtain purified glycerol.

10. A method according to claim 9, wherein the time for reaction is less than 120 minutes.

11. A method according to claim 9, wherein a pre-coat or body feed is applied to the filter.

12. A process for purifying crude glycerol, which process comprises mixing crude glycerol with CaO in a final concentration of at least 2.0 g CaO per kg of the mixture, followed by filtering the reaction mixture over a pressure filter to obtain purified glycerol.
